(19) United States
(12) Patent Application Publication
(10) Pub. No.: US 2005/0216291 A1
(43) Pub. Date: Sep. 29, 2005

(54) Complete mold system

(76) Inventors: Elias A. Shaheen, (US); Jennifer Fung, (US); Julie Wiesman, (US)

Correspondence Address: THE CLOROX COMPANY, 1221 BROADWAY PO BOX 2351, OAKLAND, CA, 94623, US

(21) Appl. No.: 10/870,096

(22) Filed: Jun. 16, 2004

Related U.S. Application Data
(63) Continuation-in-part of application No. 10/838,571, filed on May 4, 2004.
Application No. 10/838,571 is a continuation-in-part of application No. 10/806,522, filed on Mar. 23, 2004.

(51) Int. Cl. G06F 17/60

(52) U.S. Cl. 705/1

(57) ABSTRACT

This invention relates to a complete mold system that provides consumers with tools for understanding, detecting, removing and preventing mold. The complete mold system will provide consumers with one comprehensive resource for taking care of their mold problem anywhere in the home. In addition to specific tools for detecting, removing, inhibiting/delaying and preventing mold, educational materials will guide consumers in a step-by-step manner on how best to take care of their mold problem.

### CROSS REFERENCE TO RELATED APPLICATIONS

The present application is a continuation-in-part of Co-pending application Ser. No. 10/838,571, filed Apr. 23, 2004, which in turn is a continuation-in-part of co-pending application Ser. No. 10/806,522 filed Mar. 23, 2004

### BACKGROUND OF THE INVENTION

1. Field of the Invention

This invention relates to a complete mold system that provides consumers with tools for understanding, detecting, removing and preventing mold. The complete mold system will provide consumers with one comprehensive resource for taking care of their mold problem anywhere in the home. In addition to specific tools for detecting, removing, inhibiting/delaying and preventing mold, educational materials will guide consumers in a step-by-step manner on how best to take care of their mold problem.

2. Description of the Related Art

Consumers have recently become more concerned with mold due to increased media coverage of the effects of mold on health and home. In addition, research has shown that 100% of homes have mold, making mold relevant to all consumers. Although consumers know that mold is bad, they don't know how to take care of the mold problem. Several consumer products are marketed for removal of mold, however, these products do not deal with the identification and evaluation of mold or the safety requirements that may be necessary to deal with mold under certain conditions.

Mold presents special issues in treatment. Dust mite allergens, pet urine, and pet dander are non-living and, in general, are simple proteins. Mold and pollen allergens are living organisms containing protein, lipids and carbohydrates. Thus, treatments that are effective for some allergen problems may not be effective for molds and pollen.

U.S. pat. app. 2004/0020007 to Lausevic describes a vacuum cleaner with a special attachment and a HEPA filter for removing mold. U.S. Pat. No. 6,440,365 to Poye et al. describes inspecting a building for Stachybotris, applying hydrochloric acid, and heating the applied treatment. U.S. Pat. No. 5,395,541 to Carpenter et al. describes an enzyme treatment to remove glycoside-containing microorganisms.

Based on the prior art examples, various techniques have been discovered to deal with mold problems. However, the need still exists for a system to detect, remove and prevent mold problems. The complete mold system will empower consumers by providing a comprehensive solution that includes step-by-step guidelines for detecting and removing mold.

### SUMMARY OF THE INVENTION

In accordance with the above objects and those that will be mentioned and will become apparent below, one aspect of the present invention comprises system for mold removal comprising a detection device for mold and a treatment device for mold.

In accordance with the above objects and those that will be mentioned and will become apparent below, another aspect of the present invention comprises a method for providing consumers with one comprehensive resource for taking care of mold problems in the home, said method comprising the steps of: optionally, providing educational material about mold; optionally, providing a detection device for mold; optionally, providing guidelines for how to take care of the mold problem based on results of the mold detection device; optionally, providing components for treatment of mold; and optionally, providing a treatment for inhibiting future mold growth.

In accordance with the above objects and those that will be mentioned and will become apparent below, another aspect of the present invention comprises a system for mold removal comprising instructions for mold treatment, said instructions comprising the steps of: using a means for identifying the existence of mold; applying a composition for the treatment of mold; and optionally, measuring the result of the mold treatment.

In accordance with the above objects and those that will be mentioned and will become apparent below, another aspect of the present invention comprises a method of providing a system of selecting a treatment for mold to a consumer, said method comprising the steps of: optionally, collecting data on the mold conditions in the consumer's household; optionally, providing educational materials, wherein said educational materials provide information concerning the health effects of mold; providing an array of products for the treatment of mold; and providing information to said consumer regarding said products which make up said array, thereby enabling said consumer to select suitable products for the treatment of mold.

Further features and advantages of the present invention will become apparent to those of ordinary skill in the art in view of the detailed description of preferred embodiments below.

### DETAILED DESCRIPTION

Before describing the present invention in detail, it is to be understood that this invention is not limited to particularly exemplified systems or process parameters that may, of course, vary. It is also to be understood that the terminology used herein is for the purpose of describing particular embodiments of the invention only, and is not intended to limit the scope of the invention in any manner.

All publications, patents and patent applications cited herein, whether supra or infra, are hereby incorporated by reference in their entirety to the same extent as if each individual publication, patent or patent application was specifically and individually indicated to be incorporated by reference.

It must be noted that, as used in this specification and the appended claims, the singular forms “a,” “an” and “the” include plural referents unless the content clearly dictates otherwise. Thus, for example, reference to a “surfactant” includes two or more such surfactants.

Unless defined otherwise, all technical and scientific terms used herein have the same meaning as commonly understood by one of ordinary skill in the art to which the invention pertains. Although a number of methods and materials similar or equivalent to those described herein can be used in the practice of the present invention, the preferred materials and methods are described herein.

In the application, effective amounts are generally those amounts listed as the ranges or levels of ingredients in the descriptions, which follow hereto. Unless otherwise stated, amounts listed in percentage (“%'s”) are in weight percent (based on 100% active). For compositions on substrates the weight percent is of the cleaning composition alone, not accounting for the substrate weight, unless otherwise noted.

As used herein, the term “substrate” is intended to include any web, which is used to clean an article or a surface. Examples of cleaning sheets include, but are not limited to, mitts, webs of material containing a single sheet of material which is used to clean a surface by hand or a sheet of material which can be attached to a cleaning implement, such as a floor mop, handle, or a hand held cleaning tool, such as a toilet cleaning device.

As used herein, “wiping” refers to any shearing action that the substrate undergoes while in contact with a target surface. This includes hand or body motion, substrate-implement motion over a surface, or any perturbation of the substrate via energy sources such as ultrasound, mechanical vibration, electromagnetism, and so forth.

The term “cleaning composition”, as used herein, is meant to mean and include a cleaning formulation having at least one surfactant.

The term “surfactant”, as used herein, is meant to mean and include a substance or compound that reduces surface tension when dissolved in water or water solutions, or that reduces interfacial tension between two liquids, or between a liquid and a solid. The term “surfactant” thus includes anionic, nonionic and/or amphoteric agents.

Complete Mold System

The mold system can contain a combination of elements including: a mold detection device for collecting and analyzing mold presence in the home; detailed guidelines for how to take care of the mold problem based on results of the detection; components for removing or treating mold; components for ongoing mold prevention; and educational material about mold. The mold system might be part of a home construction kit targeting the bathroom. The mold system might be part of educational materials on how to maintain your home. The mold system might be part of a larger enterprise and could be expanded or broadened based on potential partnerships with (but not limited to) home insurers, property managers, professional mold remediation companies, health insurers, pharmaceutical companies, health-industry agencies (e.g. allergy associations) and government agencies (e.g. EPA, CA IAQ). The mold system may be provided in a satellite shop at the location selected from the group consisting of substantially within an existing retail store, substantially adjacent to an existing retail store, and a combination thereof.

Mold Detection Device

The mold system can contain a mold detection device for collecting and analyzing mold presence in the home. The detection device may perform quantitative and qualitative testing. For example, the detection device may verify the presence of mold, the type and level of mold present. Examples of suitable detection devices include PCT App. WO03/031562 to Green et al., PCT App. WO2004/029216 to Han et al., U.S. Pat. No. 6,713,298 to McDevitt et al., U.S. Pat. No. 5,827,748 to Golden, U.S. Pat. No. 5,858,804 to Zanzucchi et al., U.S. Pat. No. 6,146,593 to Pinkel et al., U.S. Pat. No. 5,994,149 to Robinson et al., U.S. Pat. No. 6,729,196 to Moler et al., U.S. Pat. No. 6,303,316 to Kiel et al., and U.S. Pat. No. 6,192,168 to Feldstein et al., each incorporated herein by reference in their entirety.

The detection device may be based on a biosensor. A requirement for the biosensor may be that it is capable of detecting binding of an analyte to each binding moiety spot. The detection device may perform the analysis of a fluid containing one or more analytes. The device may be used for either liquid or gaseous fluids.

A biosensor is an apparatus that uses specific and/or selective binding interactions with one or more biomolecules (“ligands”), such as peptides, proteins, enzymes, antibodies, receptors, nucleic acids, aptamers, etc. to detect one or more target molecules (“analytes”). Binding of the target molecule to the ligand results in a signal that can be used to detect or quantify the analyte present in a sample. A wide variety of biosensors of different design are known. Typically, these are designed for use in clinical or research laboratories and tend to be very bulky and relatively fragile. For example, U.S. Pat. No. 6,258,606 discloses a multiplexed active biologic electrode array, allowing a variety of protein or nucleic acid biomolecules to be attached to specific locations on an integrated circuit chip. The biomolecules are exposed to samples and binding of various analyses to specific locations on the chip may be detected, for example, by fluorescence spectroscopy. U.S. Pat. No. 6,294,392 discloses a flow-through microchannel (capillary) biosensor that is said to be suitable for the detection of multiple different analyses in a sample by binding to complementary biomolecules immobilized on the wall of the microchannel. Following initial binding, immobilized complexes are denatured and flow past a downstream detector. U.S. Pat. No. 6,171,238 discloses a portable hand-held biosensor device for examination of whole blood, urine and other biological liquids. The system contains a single measuring electrode that can be covered by a biodiaphragm, limiting detection to single analyses at a time. U.S. Pat. No. 6,192,168 discloses a multimode waveguide device and fluidics cube apparatus that may be used as a biosensor. The waveguide may be attached to different biomolecules for detecting various analyses and may contain multiple channels for processing more than one sample at a time.

The biosensor of the detection device recognizes analytes meaning any compound, molecule or aggregate of interest for detection using the biosensor. Non-limiting examples of analyses include a protein, peptide, carbohydrate, polysaccharide, lipid, hormone, growth factor, cytokine, receptor, antigen, allergen, antibody, substrate, metabolite, cofactor, inhibitor, drug, pharmaceutical, nutrient, toxin, poison, explosive, pesticide, chemical warfare agent, biowarfare agent, biohazardous agent, infectious agent, prion, radioisotope, vitamin, heterocyclic aromatic compound, carcinogen, mutagen, narcotic, amphetamine, barbiturate, hallucinogen, waste product, contaminant, heavy metal or any other molecule or atom, without limitation as to size. Analytes are not limited to single molecules or atoms, but may also comprise complex aggregates, such as a virus, bacterium, Salmonella, Streptococcus, Legionella, E. coli, Giardia, Cryptosporidium, Rickettsia, spore, mold, yeast, algae, amoebae, dinoflagellate, unicellular organism, pathogen or cell. In certain embodiments, cells exhibiting a particular characteristic or disease state, such as a cancer cell, may be target analytes. Virtually any chemical or biological compound, molecule or aggregate could be a target analyte.

In various embodiments, the present invention concerns the use of binding moieties for the detection of analytes. Although in preferred embodiments the binding moieties are antibodies, it is contemplated within the scope of the invention that virtually any molecule or aggregate that can bind to a target analyte with sufficient affinity and specificity to allow its detection may be used. Non-antibody binding moieties that may be used within the scope of the present invention include, for example, aptamers (e.g., U.S. Pat. No. 5,843,653 to Gold et al.), peptide libraries (e.g., U.S. Pat. No. 6,068,829 to Ruoslahti et al., incorporated herein by reference), and various receptor proteins, binding proteins, cell surface proteins, and other non-antibody peptides or proteins known in the art.

The terms “detection” and “detecting” are used herein to refer to an assay or procedure that is indicative of the presence of one or more specific analytes in a sample, or that predicts a disease state or a medical or environmental condition associated with the presence of one or more specific analyses in a sample. It will be appreciated by those of skill in the art that all assays exhibit a certain level of false positives and false negatives. Even where a positive result in an assay is not invariably associated with the presence of a target analyte, the result is of use as it indicates the need for more careful monitoring of an individual, a population, or an environmental site. An assay is diagnostic of a disease state or a medical or environmental condition when the assay results show a statistically significant association or correlation with the ultimate manifestation of the disease or condition.

The specimen might be sent for analysis to an offsite laboratory. The results of the test and/or the treatment guidelines might be provided over a computer communications network such as the internet, for example, as described in U.S. pat. app. 2002/0055176 to Ray. The method of providing test results and/or treatment guidelines might include a step of providing a Web page that is adapted to allow a person to enter the unique code onto the Web page and transmit an electronic message containing the unique code from a first computer communication network access device remotely-located from the off-site laboratory over the computer communications network to a second computer communication network access device located at the off-site laboratory. The computer located at the off-site laboratory can receive the electronic message containing the unique code and respond by transmitting an electronic message containing the test results over the computer communications network to the first computer communication network access device.

Mold Treatment Guidelines

The mold system can contain detailed guidelines for how to take care of the mold problem based on results of the detection kit. For example, if the type and level of mold present is below a certain hurdle, the consumer might be directed to remove the mold using additional tools in the system. Step-by-step instructions would guide the consumer on how to remove the mold. If the type and level of mold present is above a certain threshold, the consumer might be referred to a professional mold remediation company, who might have a relationship with the mold system provider.

The mold system can contain detailed guidelines for evaluating buildings for mold growth. Such instructions might include: “Check building materials and spaces for visible mold and signs of moisture damage indicating a history of water leaks and high humidity and condensation levels. Building ventilation systems should also be inspected. Basic precautions should be taken when investigating and evaluating mold and moisture problems. Such precautions could include: Do not touch mold or moldy items with bare hands; Do not get mold or mold spores in your eyes; Do not breathe mold or mold spores; Use personal protective equipment (PPE). At a minimum, use an N-95 NIOSH-approved respirator, gloves, and eye protection; and Contain or bag debris.”

Sampling instructions might include: “Sampling is usually not necessary when visible signs of mold growth are present. However, the American Industrial Hygiene Association (AIHA) indicates that in cases where health concerns are an issue, litigation is involved, or the source(s) of contamination is unclear, sampling may be considered. Professionals experienced with mold issues and familiar with current guidelines should conduct sampling and interpret results, as no threshold or exposure limits have been established. As a general,guideline, the types and concentrations of mold in indoor air samples should be similar to those found in the local outdoor air. Samples should be analyzed by a laboratory that participates in proficiency testing such as the Environmental Microbiology Proficiency Analytical Testing Program, EMPAT.”

Remediation instruction might include: “Mold remediation prevents further human exposure and damage to building materials and furnishings. You must clean up and remove mold contamination, not just kill the mold. Dead mold is still allergenic; some are potentially toxic. Mold gradually destroys what it grows on; to grow, it needs an organic substrate, moisture, and oxygen. If mold growth is not addressed promptly, materials may be damaged and cleaning cannot restore appearance or integrity. Mold can generally be removed from nonporous (hard) surfaces by wiping or scrubbing with water or water and detergent. The use of disinfectant chemicals (biocides), including chlorine bleach, is not recommended as a routine practice. Biocides are of limited use in mold remediation and are not a substitute for thorough cleaning. Mold-contaminated porous material such as damp insulation in ventilation systems, moldy ceiling tile, and mildewed carpet may need to be removed and discarded. Remediate means to fix a problem. The first step in mold remediation is to fix the water or humidity problem that contributed to mold growth. Thoroughly clean up mold and dry water-damaged areas, using appropriate cleaning and drying methods. Mold remediation requires some level of isolation of materials or containment and the use of appropriate personal protective equipment (PPE). Remediation decisions should be based on the scope of contamination, size of the area of growth, and potential for occupant exposure or building contamination in the absence of containment. Professional expertise and conservative methods may be needed when the chance of mold becoming airborne is high or mold-sensitive individuals are present.”

Cleanup methods might include: “Small—less than 10 sq. ft. Example: Carpet and backing. Wet vacuum. Use high-efficiency particulate air (HEPA) vacuum when thoroughly dry. Medium—10-100 sq. ft. Example: Concrete or cinder block. Wet vacuum. Use HEPA vacuum when thoroughly dry. Large—greater than 100 sq. ft. Example: Drywall or gypsum. Use HEPA-vacuum after thoroughly dry. Remove and discard damaged material.”

Components for Removing and Treating Mold

The mold system can contain components for removing mold. Suitable components might include: disposable gloves to prevent physical contact of the skin with mold; a disposable mask to prevent inhalation of mold spores; a pre-moistened wipe with diluted bleach to remove, kill and denature mold; a traditional hypochlorite spray product to remove and kill mold; an aerosol spray product to remove and kill airborne and surface mold; and a calorimetric Indicator to confirm cleaning and disinfecting process is successful. The mold system can also contain such items as mold-resistant grout, a tool for applying grout, a tool for removing old grout. Additional equipment required might include a N-95 respirator, goggles/eye protection, disposable overalls, and a HEPA-filtered fan unit.

Various components can be included in the mold system for treating mold in the home. For example, U.S. 2004/0001777 to Hobson et al. describes evaporating a solution of acidified oxyhalogen species. The treatment may be provided by filters such an HEPA filters, for example, as described in U.S. pat. app. 2003/0150327 to Bolden. The treatment may be provided by electrostatic filters, for example, as described in U.S. Pat. No. 6,656,253 to Willey et al. The treatment may provide a variety of treatment mechanisms, for example, as described in U.S. pat. app. 2004/0047776 to Thomsen. The treatment may provide a chemical means to decontaminate, for example, U.S. pat. app. 2003/0056648 to Fomai et al. The chemical means may be a source of active material from the group consisting of hypohalous acid, hypohalous acid salt, hypohalous acid generating species, hypohalous acid salt generating species, and combinations thereof. The treatment may be provided by typical chemical compositions or cleaning substrates, for example, U.S. pat. app. 2003/0228996 to Hei et al., U.S. Pat. No. 6,576,604 to Hoshino et al., U.S. Pat. No. 6,200,941 to Strandburg et al., U.S. Pat. No. 5,972,864 to Counts, U.S. Pat. No. 5,972,239 to Coyle-Rees, U.S. Pat. No. 5,929,013 to Kuriyama et al., U.S. Pat. No. 5,869,440 to Kobayashi et al., U.S. Pat. No. 5,783,550 to Kuriyama et al., U.S. pat. app. 2004/0072712 to Man et al., U.S. Pat. No. 5,688,756 to Garabedian et al., U.S. Pat. No. 6,624,134 to Briatore et al., Co-pending Application (Docket No. 340.182), which was filed Mar. 23, 2004, entitled “Methods for deactivating allergens and preventing disease”, Co-pending application Ser. No. 10/632573, which was filed Aug. 1, 2003, entitled “Disinfecting Article With Extended Efficacy”, and Co-pending application Ser. No. 10/828571, which was filed Apr. 23, 2004, entitled “Method for Diluting Hypochlorite”.

Educational Materials About Mold

The system may provide educational material about mold, including but not limited to technical information and pictures of common household mold, health effects of exposure to mold, preventive measures, tips on cleaning the home and maintaining a “healthy home”.

An example of educational information about mold includes the following statements. Molds are usually not a problem indoors, unless mold spores land on a wet or damp spot and begin growing. Molds have the potential to cause health problems. Molds produce allergens (substances that can cause allergic reactions), irritants, and in some cases, potentially toxic substances (mycotokins). Inhaling or touching mold or mold spores may cause allergic reactions in sensitive individuals. Allergic responses include hay fever-type symptoms, such as sneezing, runny nose, red eyes, and skin rash (dermatitis). Allergic reactions to mold are common. They can be immediate or delayed. Molds can also cause asthma attacks in people with asthma who are allergic to mold. In addition, mold exposure can irritate the eyes, skin, nose, throat, and lungs of both mold-allergic and non-allergic people. Molds can also produce organic toxins. These toxins include Aflatoxin B, Citrinin, Cyclosporin A, Deoxynivalenol, Emodin, Gliotoxin, Griseofulvin, Ochratoxin A, Patulin, Roridin A, Satratoxin H, Sterigmatocystin, T-2 toxin, Verrucarin A, and Endotoxins. Molds are living organisms containing protein, lipids and carbohydrates. Thus, treatments that are effective for some chemicals may not be effective for molds. The use of a chemical or biocide that kills organisms such as mold is not recommended as a routine practice during mold cleanup. Dead mold may still cause allergic reactions in people, so it is not enough to simply kill the mold, it must also be destroyed or removed.

The first step in the educational materials might allow consumers to identify where they have a mold problem and gauge the magnitude of their problem. The educational materials might include where to look for mold; such as, “Mold grows on organic materials, such as paper, dirt, wood and soap scum. Mold grows on moist materials, so mold growth is likely in areas wet by water leaks, flooding, humidity levels above about 70 percent and condensation. Any flooded area that was not completely dried within about one day is likely to have mold growth. Walls need to be opened and rapidly dried to prevent mold growth. Any area that is stained from water should be examined for mold growth. Peeling paint may be an indication of wet walls. Moisture seeping through concrete walls and floors will cause moist conditions likely to cause mold growth on or in walls, carpeting and materials stored in the basement. Mold often grows under cabinets, behind base-boards, inside walls, in carpet padding and under vinyl wall coverings. An unvented clothes dryer creates a very humid, warm environment conducive to mold growth. Closets may have mold growth if clothing is damp or if there is a cool outside wall in the closet. Also, there is a chance mold might be growing behind furniture, particularly against an outside wall. Mold will not normally be found in furnace or air-conditioning ducts unless they were flooded because the heated or air-conditioned air is very dry. Moisture coming through a basement floor or wall may deposit a light-colored salt and other minerals that are sometimes thought to be mold. The deposits should quickly dissolve and disappear when wet with water if they are a salt.”

The educational materials might include directions for mold removal; such as, “Since people react to mold whether it is living or dead, the mold must be removed. Take steps to protect your health during mold removal. Use a mask or respirator that will filter out mold spores. Usually it will be designated as an N95, 3M #1860 or TC-21C particulate respirator. Wear eye protection, rubber gloves and clothing that can be immediately laundered. Dampen moldy materials before removal to minimize the number of airborne mold spores. Mold can be removed from hard surfaces such as hard plastic, glass, metal and counter tops by scrubbing with a non-ammonia soap or detergent.. It is impossible to completely remove mold from porous surfaces such as paper, Sheetrock (drywall) and carpet padding, so these materials should be removed and discarded. Scrubbing may not completely remove mold growth on structural wood, such as wall studs, so it may need to be removed by sanding. Wear personal protective gear and isolate the work area from the rest of the home. After the mold is removed, disinfect the area using a bleach and water solution or another disinfectant. The amount of bleach recommended per gallon of water varies considerably. A clean surface requires less bleach than a dirty surface. A solution of ¼ cup bleach to 1 gallon of water should be adequate for clean surfaces. The surface must remain wet for about 15 minutes to allow the solution to disinfect. Concentrations as high as ½ cups of bleach per gallon of water are recommended for surfaces that could not be thoroughly cleaned. Provide adequate ventilation during disinfecting and wear rubber gloves. Finally, rinse the entire area with clean water, and then rapidly dry the surfaces. Use fans and dehumidifiers or natural ventilation that exchanges inside air with outside air.”

The educational materials might include directions for preventing mold growth; such as, “The moisture problem must be fixed to prevent future mold growth. Since there are some mold spores everywhere and since mold grows on any wet organic surface, the only way to prevent mold growth is to keep things dry.”

The mold educational materials could include government materials, such as EPA's pamphlet, “Mold Remediation in Schools and Commercial Buildings.” It provides clean-up methods and remediation techniques and discusses precautions and the impact of mold on HVAC systems. Its guidelines are based on total surface area contamination and potential for remediator and occupant exposure. The mold educational materials could include referral to internet websites for additional information, such as www.epa.gov/iaq/molds and www.osha.gov/SLTC/molds.

Treatment for Inhibiting Future Mold Growth

Chemical treatments have been developed for residual mold control, for example, PCT App. WO02/28990 to McKechnie; U.S. Pat. No. 6,559,111 to Colurciello et al., and U.S. pat. app. 2002/0193278 to Cermenati et al. Surface treatments have been developed for residual mold control, for example, PCT App. WO2002/664877 to Rohrbaugh et al., U.S. pat. app. 2003/0171446 to Murrer et al., and U.S. pat. app. 2002/0028288 to Rohrbaugh et al. Devices that have been developed for residual mold control include U.S. pat. app. 2003/0032569 to Takemura et al. and U.S. Pat. No. 6,463,600 to Conway et al.

Method of Use

The treatment composition may be dispersed into the air. The composition may be dispersed by an atomizer, a vaporizer, a nebulizer, or a spray device. The composition may be delivered on a continuous basis, such as with a humidifier. The composition may be delivered on a pulsed basis, such as with a canister on a timer. One spray device is an electrostatic sprayer, as described in WO0120988. The composition may be applied to skin surfaces. The compositions may be contained within a treatment device.

The composition may be applied to soft surfaces including clothing, bedding, upholstery, curtains, and carpets. The composition may be applied to soft surfaces by spraying, by wiping, by direct application, by immersion, or as part of the laundry washing process.

The composition may be applied to hard surfaces including kitchen surfaces, bathroom surfaces, walls, floors, outdoor surfaces, automobiles, countertops, food contact surfaces, toys, food products including fruits and vegetables. The composition may be applied to hard surfaces by spraying, by wiping, by direct application, by immersion, or as part of the normal cleaning process.

The composition may be applied with a nonwoven substrate, wipe or cleaning pad on inanimate, household surfaces, including floors, counter tops, furniture, windows, walls, and automobiles. The composition may be applied to baby and children's items, including toys, bottles, pacifiers, etc. Other surfaces include stainless steel, chrome, and shower enclosures. The nonwoven substrate, wipe or cleaning pad can be packaged individually or together in canisters, tubs, etc. The nonwoven substrate, wipe or cleaning pad can be used with the hand, or as part of a cleaning implement attached to a tool or motorized tool, such as one having a handle. Examples of tools using a nonwoven substrate, wipe or pad include U.S. Pat. No. 6,611,986 to Seals, WO00/71012 to Belt et al., U.S. pat. app. 2002/0129835 to Pieroni and Foley, and WO00/27271 to Policicchio et al.

For certain uses, the composition may be thickened. The composition may be thickened by surfactant thickening, polymer thickening, or other means. Thickening may allow more controlled application or application from a device. Examples of thickened and unthickened compositions can be found in U.S. Pat. No. 6,162,371, U.S. Pat. No. 6,066,614, U.S. Pat. No. 6,153,120, U.S. Pat. No. 6,037,318, U.S. Pat. No. 6,313,082, U.S. Pat. No. 5,688,435, U.S. Pat. No. 6,413,925, U.S. Pat. No. 6,297,209, U.S. Pat. No. 6,100,228, U.S. Pat. No. 5,916,859, U.S. Pat. No. 5,851,421, U.S. Pat. No. 5,688,756, U.S. Pat. No. 5,767,055, U.S. Pat. No. 5,055,219, and U.S. Pat. No. 5,075,029.

The composition may be prepared by mixing a solid composition with water. The solid composition may be a tablet, granular composition, paste, or other solid composition. The composition may be prepared by diluting a liquid composition with water. The water may be purified. The composition may be prepared by mixing two liquids, for example, from a dual chambered container or a dual chambered spray bottle. The compositions of the invention can be delivered as part of a multi-compartment delivery system, for example as described in U.S. Pat. No. 5,954,213, U.S. Pat. No. 5,316,159, WO2004/014760, U.S. Pat. No. 6,610,254, and U.S. Pat. No. 6,550,694.

The composition may be part of an article of manufacture, wherein said article of manufacture in addition to the usage instructions bears an additional indication comprising a term selected from the group consisting of: neutralizes mold allergens, denatures toxins from mold, neutralizes toxins from mold, neutralizes protein allergens, controls allergens, removes allergens by cleaning, removes allergens by wiping, removes allergens in the laundry, reduces respiratory illness, reduces hay fever, reduces absenteeism, denatures mold allergens, prevents allergenic reactions, prevents allergenic reaction in humans, prevents allergenic symptoms due to mold, kills mold, destroys mold spores, destroys mold spores that cause adverse health effects, proven to prevent mold-triggered allergic sensitization in humans, proven to prevent mold-triggered allergic sensitization in animals, reduces the risk of mold-triggered allergic sensitization, reduces the risk of mold-triggered allergic response, destroys mold spores that induce allergic symptoms, neutralizes mold specific antigens, and prevents non-immune inflammatory reactions to mold.

The article of manufacture may include a set of instructions. The set of instructions may be used with a method of instructing the public by providing to the public a set of instructions for the use of an article of manufacture. The method of instructing the public may include information that an allergic response represents a response to pollen, dust mite, or mold allergens. The set of instructions may be provided to the public via electronic and/or print media. The set of instructions may be posted at the point of sale adjacent the package. The set of instructions may be posted on a global computer network at an address associated with products from a group consisting of said liquid composition, said target surface, or a combination thereof.

This invention has been described herein in considerable detail to provide those skilled in the art with information relevant to apply the novel principles and to construct and use such specialized components as are required. However, it is to be understood that the invention can be carried out by different equipment, materials and devices, and that various modifications, both as to the equipment and operating procedures, can be accomplished without departing from the scope of the invention itself. As such, these changes and modifications are properly, equitably, and intended to be, within the full range of equivalence of the following claims.

1. A system for mold removal comprising:
a. a detection device for mold; and
b. a treatment device for mold.

2. The system of claim 1, wherein said treatment device comprises chemical treatment.

3. The system of claim 2, wherein said chemical treatment comprises a source of active material from the group consisting of hypohalous acid, hypohalous acid salt, hypohalous acid generating species, hypohalous acid salt generating species, and combinations thereof.

4. The system of claim 1, wherein said treatment device comprises a filter.

5. The system of claim 1, wherein said detection device is portable.

6. The system of claim 1, wherein said detection device at least partially operates at a remote location.

7. The system of claim 6, wherein said remote location provides test results over a computer communications network.

8. The system of claim 1, wherein said detection device comprises a biosensor.

9. The system of claim 1, wherein said detection device identifies a particular analyte selected from the group comprising a particular mold and a particular mold byproduct.

10. A method for providing consumers with one comprehensive resource for taking care of mold problems in the home, said method comprising the steps of:
a. optionally, providing educational material about mold;
b. optionally, providing a detection device for mold;
c. optionally, providing guidelines for how to take care of the mold problem based on results of the mold detection device;
d. optionally, providing components for treatment of mold; and
e. optionally, providing a treatment for inhibiting future mold growth.

11. The method of claim 10, wherein said components are selected from the group consisting of tools, chemical products, protective devices, treatment devices, instructions, and combinations thereof.

12. The method of claim 10, wherein said method is part of a kit for home remodeling.

13. The method of claim 10, wherein said method is part of educational materials on how to maintain your home.

14. The method of claim 10, wherein said method additionally includes instructions to contact an outside group, such as a mold remediation company.

15. A system for mold removal comprising instructions for mold treatment, said instructions comprising the steps of:
a. using a means for identifying the existence of mold;
b. applying a composition for the treatment of mold; and
c. optionally, measuring the result of the mold treatment.

16. The system according to claim 15, wherein said instructions are provided by means of a leaflet or printed matter which is separate from said composition and is separate from said means of identifying the existence of mold.

17. A method of providing a system of selecting a treatment for mold to a consumer, said method comprising the steps of:
a. optionally, collecting data on the mold conditions in the consumer's household;
b. optionally, providing educational materials, wherein said educational materials provide information concerning the health effects of mold;
c. providing an array of products for the treatment of mold; and
d. providing information to said consumer regarding said products which make up said array, thereby enabling said consumer to select suitable products for the treatment of mold.

18. The method of claim 17, wherein said data is collected via an interactive mechanism.

19. The method of claim 17, wherein said interactive mechanism comprises a computer.

20. The method of claim 17, wherein said educational materials comprise information on health effects of exposure to mold.

21. The method of claim 17, wherein said method is provided in a satellite shop at the location selected from the group consisting of substantially within an existing retail store, substantially adjacent to an existing retail store, and a combination thereof.
